(19) United States
(12) Patent Application Publication
(10) Pub. No.: US 2015/0126822 A1
(43) Pub. Date: May 7, 2015

(54) CONGESTIVE HEART FAILURE RISK STATUS DETERMINATION METHODS AND RELATED DEVICES

(71) Applicant: Medtronic Monitoring, Inc., San Jose, CA (US)

(72) Inventors: Abhi CHAVAN, Maple Grove, MN (US); Niranjan CHAKRAVARTHY, Eden Prairie, MN (US)

(21) Appl. No.: 14/529,717

(22) Filed: Oct. 31, 2014

Related U.S. Application Data
(60) Provisional application No. 61/898,624, filed on Nov. 1, 2013.

(51) Int. Cl. A61B 5/0205; A61B 5/04; A61B 5/00; A61B 5/02; A61B 5/0402; A61B 5/11

(52) U.S. Cl. CPC A61B 5/0205; A61B 5/0402; A61B 5/1121; A61B 5/1118; A61B 5/4875; A61B 5/02028; A61B 5/04017; A61B 5/1116; A61B 5/4806; A61B 5/7246; A61B 5/6833; A61B 5/0006; A61B 5/053
USPC 600/301; 600/300; 600/509; 600/547; 600/595; 600/483; 600/508; 600/529; 600/538; 600/484; 600/391

(57) ABSTRACT

Embodiments relate to devices and methods for monitoring, identifying, and determining risk of congestive heart failure (CHF) hospitalization. Methods include determining physiological values of a patient by electrocardiogram (ECG), bioimpedance, and 3-axis accelerometer, filtering the physiological values, comparing physiological values to baseline parameters and determining CHF risk. Devices include a 3-axis accelerometers, bioimpedance sensors, and an electrocardiogram, each capable of measuring patient physiological values, and one or more processors to receive the measured physiological values.

### BACKGROUND

A number of physiological parameters may be measured in the medical industry for patient diagnosis, treatment, and monitoring. Electrocardiogram (ECG) measurements may be used to determine physiological parameters such as heart rate and heart rhythm. Bioimpedance measurements may be used to determine physiological parameters, such as, for example, fluid status, breath rate and breath volume. Accelerometer measurements may be used to determine physiological parameters, such as, for example, activity intensity, activity duration, and patient posture.

Physiological parameters can be used to diagnose and/or monitor medical conditions such as congestive heart failure (CHF) during and/or after hospitalization. However, measured physiological parameters may not always be reliable for the purposes of monitoring patient CHF health status. For example, heart rate measurements may be unreliable for purposes of CHF diagnosis during periods of irregular, yet potentially healthy activity. In some cases measured physiological parameters may be compared to baseline physiological parameters of a patient or a patient population. However, individual patient lifestyles or physical characteristics can render comparisons of measured physiological values to a baseline of a patient or patient population values unreliable for the purposes of monitoring CHF health status. Post-hospitalization CHF monitoring programs may require implantable or cumbersome measurement devices which may prevent optimum patient compliance and participation. Current CHF monitoring techniques may be less than effective at reducing CHF re-hospitalization rates.

### SUMMARY

In general, this disclosure describes techniques for determining a worsening CHF status using patient physiological parameters measured by a device. In particular, this disclosure describes techniques for monitoring and determining a risk of hospitalization as a result of congestive heart failure (CHF). It should be noted that although the techniques of this disclosure are described with respect to examples for monitoring and determining CHF status and hospitalization risk, the techniques described herein are generally applicable to monitoring and determining the risk of hospitalization due to other medical conditions.

According to one example of the disclosure, a method for monitoring and determining CHF hospitalization risk comprises determining one or more physiological values of a patient, determining whether the patient is at rest, filtering the one or more physiological values based on a patient rest state, and determining CHF risk based on the one or more filtered physiological values. Physiological values can include fluid status, heart rate, heart rhythm, breath rate, and breath volume, activity intensity, activity duration, and posture, and can be determined by obtaining electrocardiogram (ECG) recordings, bioimpedance measurements, and 3-axis accelerometer measurements, for example.

According to another example of the disclosure, determining whether a patient is at rest comprises obtaining 3-axis accelerometer measurements, wherein an x-axis and a y-axis are on the patient's body plane and a z-axis is perpendicular to the body plane to determine patient activity and posture, and determining one or more of a distribution range of z-axis measurements, a relative magnitude of the z-axis measurements to the x-axis measurements, and the y-axis measurements, and a relative magnitude of the z-axis measurement to a sum of the squared x-axis measurements and squared y-axis measurements.

According to another example of the disclosure, filtered physiological values include fluid status measured while a patient is at rest, fluid status measured while a patient is not at rest, heart rate measured while the patient is at rest, heart rhythm measured while the patient is at rest, heart rhythm measured while the patient is not at rest, breath rate measured while the patient is at rest, breath volume measured while the patient is at rest, activity intensity measured while the patient is not at rest, activity duration measured while the patient is not at rest, patient posture measured while the patient is at rest, and patient posture measured while the patient is not at rest.

According to another example of the disclosure, determining CHF risk comprises comparing filtered physiological values to baseline physiological values to generate a comparison output, wherein baseline values are determined based on one or more of patient demographics, physiological values determined during a patient hospitalization, historic physiological values determined after a patient hospitalization, clinical lab results, and patient medical history. Comparison outputs can comprise one or more of an occurrence flag indicating that a filtered physiological value is below or exceeds a baseline or threshold physiological value, or a comparative percentage to which filtered physiological values are below or exceed a baseline or threshold physiological value.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 illustrates a schematic view of a monitoring and treatment system comprising a patient utilizing a medical device measuring one or more physiological values, according to one or more techniques of this disclosure.

FIG. 2A and FIG. 2A1 show an exploded view and a side cross-sectional view, respectively, of examples of the adherent device with a temperature sensor affixed to the gel cover, according to one or more techniques of this disclosure.

FIG. 2B shows a printed circuit board and electronic components over the adherent patch, as in FIG. 2A, according to one or more techniques of this disclosure.

FIG. 3A illustrates a block flow diagram of a method of determining patient CHF risk using physiological measurements, according to one or more techniques of this disclosure.

FIG. 3B illustrates a block flow diagram of a method of determining patient CHF risk using baseline physiological values, according to one or more techniques of this disclosure.

FIG. 4 illustrates a bar chart showing a sample distribution of comparison output indicator flags for heart failure patients and non-heart failure patients.

FIGS. 5A-5D illustrate analysis of filtered heart rate data in a moving window according to various techniques of this disclosure.

FIGS. 6A-6B illustrate display interfaces provided at a remote monitoring station to allow medical personnel to monitor and prioritize a plurality of patients at once according to one or more techniques of this disclosure.

### DETAILED DESCRIPTION

Congestive heart failure occurs when the heart fails to pump at a level required to provide sufficient blood flow throughout the body. Heart failure does not necessarily only describe a condition in which the heart has stopped pumping, rather it describes a decrease in performance of the heart. In some cases, persons experiencing heart failure may live relatively normal lives. In other cases, persons experiencing heart failure may require immediate medical attention such as hospitalization in order to avoid death.

Patients with a history or risk of congenital heart failure (CHF) may be monitored to determine one or more of a number of physiological values such as, for example, fluid status, heart rate, heart rhythm, breath rate, breath volume, activity duration, activity intensity, and body posture. From the determined physiological values, a CHF status may be determined. For example, a decrease in a patient's activity duration and intensity may indicate a worsening CHF status. Multiple determined physiological values may be combined to determine a CHF status. For example, a worsening CHF status with chest fluid overload and congestion is typically associated with increased fluid status and orthopnea (shortness of breath while lying down) which can cause the patient to sleep in non-supine postures, such as upright. Therefore in some cases, patient posture while sleeping can indicate a worsening CHF status.

Non-invasive monitoring and risk stratification and determination methods and devices for medical conditions and risks such as CHF have been described in U.S. Pub. No. US-2009-0076345-A1, entitled “Adherent Device with Multiple Physiological Sensors”, U.S. Pat. No. 8,473,047 issued on 25 Jun. 2013 titled “Multifrequency Bioimpedance Device and Related Methods”, and U.S. Pub. No. US-2009-0076344-A1, entitled “Multi-sensor Patient Monitor to Detect Impending Cardiac Decompensation”, the full disclosures of which are incorporated herein by reference and suitable for combination in accordance with some examples of the present invention as described herein. Such methods and devices are used to measure and filter a variety of physiological values which can be used to track physiological conditions over time and guide physicians and other health care providers in assessing and treating a patient for a variety of conditions, such as CHF.

The filtered physiological values can be displayed to the physician relative to values that are appropriate for that patient, such as baseline values or threshold values. This allows the physician to have an assessment of a patient status relative to a range of values that are appropriate and specific for that patient. Deviation from those values would signify and quantify a change in patient status—either improvement or worsening in fluid status. The stratified CHF risk information can also be displayed to the physician. In either case, the physician would then decide to hospitalize, continue monitoring, or discontinue monitoring the patient. For example, a dramatic increase in patient sedentary activity could prompt a physician to hospitalize a patient, increase monitoring, or adjust baseline values or thresholds at which the patient should be hospitalized.

Filtering of measured physiological values is required because physiology and activity of a patient can influence the measurements. Physiology of the patient can also influence the measurements. For example, fat comprising adipose tissue, fat molecules, fat cells and combinations thereof can influence impedance measurements. The fat may comprise a layer of tissue disposed under the skin that can influence the impedance measured through the skin of the patient. For example, electrical current passed through the fat tissue can increase the measured impedance of the patient. Alternatively or in combination, the fat can be disposed in the internal tissues of the patient. For example, fat molecules can permeate internal tissues and may influence impedance measurements of implanted electrodes passing an electrical current through the internal tissues or of electrodes disposed on the skin passing current through the internal tissue. For example, fat may increase the measured impedance of a patient having normal hydration such that the measured impedance is abnormally high, for example, when the patient has normal hydration and would appear dehydrated based on an impedance measurement alone.

Other physiological value filtering may be required where a device is capable of measuring more than one physiological value. For example, a 3-axis accelerometer, such as one on-board a Piix device, generates 3-axis accelerometer values wherein the x-axis and y-axis are on the patient's body plane, and the z-axis is normal or perpendicular to the body plane. The 3-axis accelerometer values are pre-processed with low-pass and high-pass filters to remove posture related offsets and transients before computing the activity intensity. Low-pass filtered values are used to compute, for example, a patient's posture-related offset trends and body posture angles over time. High-pass filtered values are used to compute, for example, a patient's activity intensity or duration.

However, physiological values may trigger an alert or warning regarding deterioration in patient health status when no such deterioration has occurred. For example, a shortness of breath resulting from healthy activity may register as a CHF indicator such as orthopnea. Accordingly, there is a need for a CHF risk determination suite which can filter input physiological values to only include those measured at reliable and appropriate times. Additionally, there is a need for methods and devices which can combine pre-test likelihood of re-hospitalization, or risk determination, with sequential diagnoses from devices such as Piix devices to generate post-test likelihood of re-hospitalization, or risk determination, due to, for example, CHF.

Embodiments of the present invention relate to methods for determining a health status and the likeliness of hospitalization or re-hospitalization of a patient, particularly as a result of CHF. Methods include determining one or more physiological values of a patient, determining whether the patient is at rest, filtering the one or more physiological values based on whether the patient is at rest, and determining CHF status. Examples also provide for devices related to the methods described herein. For example, the device may comprise electrocardiogram (ECG) circuitry capable of measuring heart rate and rhythm, an accelerometer capable of measuring patient activity and posture, and impedance circuitry capable of measuring fluid status, breath rate and volume. Additional or alternative sensors can be used. For example, breathing may be determined with a sensor that provides a signal in response to expansion of the chest and expansion of the skin of the patient.

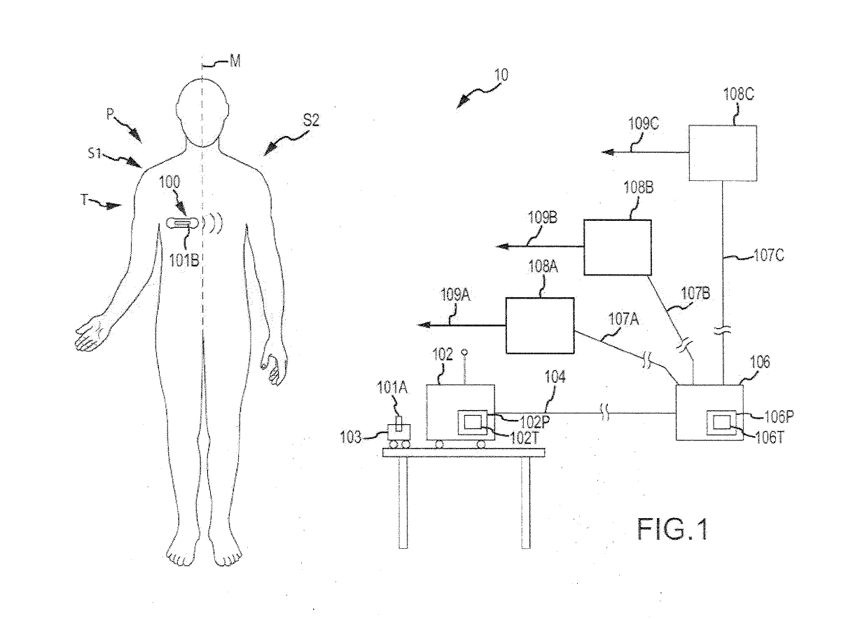

FIG. 1 shows a patient P and a monitoring system 10. Patient P comprises a midline M, a first side S1, for example a right side, and a second side S2, for example a left side. Monitoring system 10 comprises a patient measurement device to monitor the patient which may comprise an implantable device 100I or an adherent device 100, for example. Adherent device 100 can be adhered to a patient P at many locations, for example thorax T of patient P. In many embodiments, the adherent device may adhere to one side of the patient, from which side data can be collected. Work in relation with embodiments of the present invention suggests that location on a side of the patient can provide comfort for the patient while the device is adhered to the patient. The monitoring system 10 and adherent device 100 may comprise components as described in U.S. Pub. No. US-2009-0076345-A1, entitled “Adherent Device with Multiple Physiological Sensors”, and U.S. Pub. No. US-2009-0076344-A1, entitled “Multi-sensor Patient Monitor to Detect Impending Cardiac Decompensation”, the full disclosures of which are incorporated herein by reference.

Adherent device 100 can wirelessly communicate with remote center 106. The communication may occur directly (via a cellular or Wi-Fi network), or indirectly through intermediate device or gateway 102. The gateway 102 may comprise components of the zLink™, a small portable device similar to a cell phone that wirelessly transmits information received from PiiX™ to Corventis, commercially available from Corventis Inc. of San Jose, Calif. The gateway 102 may consist of multiple devices, which can communicate wired or wirelessly with remote center 106 in many ways, for example with a connection 104 which may comprise an Internet connection and/or with a cellular connection. Remote center 106 may comprise Corventis Web Services, a hosted application for data analysis and storage that also includes a website, which enables secure access to physiological trends and clinical event information for interpretation and diagnosis. Remote center 106 may further or alternatively comprise a back-end operation where physiological data from the device are read by expert human operators to verify accuracy. For example, ECG strips captured from a device can be adjudicated for arrhythmias by experts.

In many embodiments, monitoring system 10 comprises a distributed processor system with at least one processor comprising a tangible medium of device 100, at least one processor 102P of gateway 102, and at least one processor 106P at remote center 106, each of which processors can be in electronic communication with the other processors. At least one processor 102P comprises a tangible medium 102T, and at least one processor 106P comprises a tangible medium 106T. Remote processor 106P may comprise a backend server located at the remote center. Remote center 106 can be in communication with a health care provider communication device 108A with a communication system 107A, such as the Internet, an intranet, phone lines, wireless and/or satellite phone. Health care provider communication device 108A, for example for a family member, can be in communication with patient P with a communication, as indicated by arrow 109A. Remote center 106 can be in communication with a health care professional, for example with a physician 108B communication device, with a communication system 107B, such as the Internet, an intranet, phone lines, wireless and/or satellite phone. Physician communication device 108B can be in communication with patient P with communication, for example with a two way communication system, as indicated by arrow 109B. The PDA may comprise a tangible medium having instruction of a computer program embodied thereon to display the patient data to the physician. Remote center 106 can be in communication with an emergency responder device 108C, for example a communication device for a 911 operator and/or paramedic, with a communication system 107C. In many embodiments, instructions are transmitted from remote site 106 to a processor supported with the adherent patch on the patient, and the processor supported with the patient can receive updated instructions for the patient treatment and/or monitoring, for example while worn by the patient. Emergency responder device 108C can travel with the responder to the patient as indicated by arrow 109C. Thus, in many embodiments, monitoring system 10 comprises a closed loop system in which patient care can be monitored and implemented from the remote center in response to signals from the adherent device.

Each of the above described communication devices may comprise a display coupled to a processor having a tangible medium comprising a memory with instructions of a computer program embodied thereon, for example a personal digital assistant (PDA) such as a smart phone, for example a iPhone™, or Blackberry™

In many embodiments, adherent device 100 may continuously monitor physiological parameters, communicate wirelessly with a remote center, and provide alerts when necessary. The adherent patch may attach to the patient's thorax and contains sensing electrodes, battery, memory, logic, and wireless communication capabilities. In some embodiments, remote center 106 receives the patient data and applies a patient evaluation algorithm, for example the prediction algorithm to predict patient physiological or mental deterioration. In some embodiments, the algorithm may comprise an algorithm to predict impending patient physiological or mental deterioration, for example based on decreased hydration and activity. When a flag is raised, the center may communicate with the patient, hospital, nurse, and/or physician to allow for therapeutic intervention, for example to prevent further physiological or mental deterioration.

Adherent device 100 may be affixed and/or adhered to the body in many ways. For example, with at least one of the following an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide I remove old patch|place new patch|remove guide), or a keyed attachment for chatter reduction. The patch and/or device may comprise an adhesiveness embodiment (e.g. chest strap), and/or a low-irritation adhesive for sensitive skin. The adherent patch and/or device can comprise many shapes, for example at least one of a dogbone, an hourglass, an oblong, a circular or an oval shape.

In many embodiments, adherent device 100 may comprise a reusable electronics module with replaceable disposable patches, and each of the replaceable patches may include a battery. The adherent device 100 may comprise components of the Piix™, an unobtrusive, water-resistant, patient-worn device that adheres to the skin and automatically collects and transmits physiological information, commercially available from Corventis Inc. of San Jose, Calif. In some embodiments, the device may have a rechargeable module, and may use dual battery and/or electronics modules, wherein one module 101A can be recharged using a charging station 103 while the other module 101B is placed on the adherent patch with connectors. In some embodiments, the gateway 102 may comprise the charging module, data transfer, storage and/or transmission, such that one of the electronics modules can be placed in the gateway 102 for charging and/or data transfer while the other electronics module is worn by the patient.

System 10 can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, heart rate variability (HRV), heart rate turbulence (HRT), heart sounds (e.g. S3), respiratory sounds, blood pressure, activity, posture, wake/sleep, orthopnea, temperature/heat flux, and weight. The activity sensor may comprise one or more of the following: ball switch, accelerometer, minute ventilation, HR, bioimpedance noise, skin temperature/heat flux, BP, muscle noise, posture.

Patients shown may have different physical attributes, such that it can be helpful to determine the output based on the data of the patient. Each of the patients may have a body mass index determined based on the height and weight of the patient, or other personal attributes that may affect the efficacy of measured physiological values as used to determine a CHF status.

Work in relation to embodiments indicates patient characteristics can influence the measurements of the patient. For example, patient demographics such as age, gender and race can be related to the measurements of the patient.

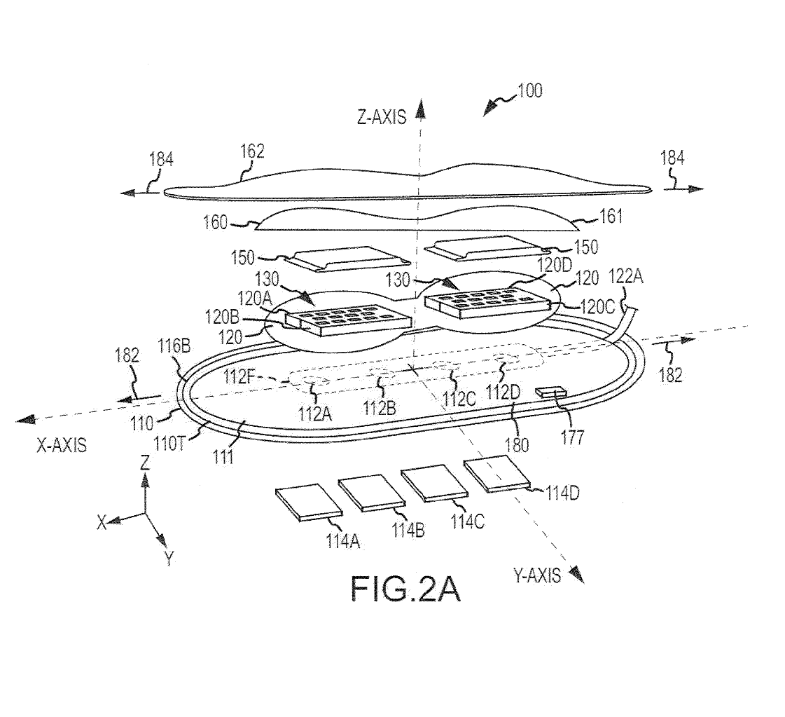

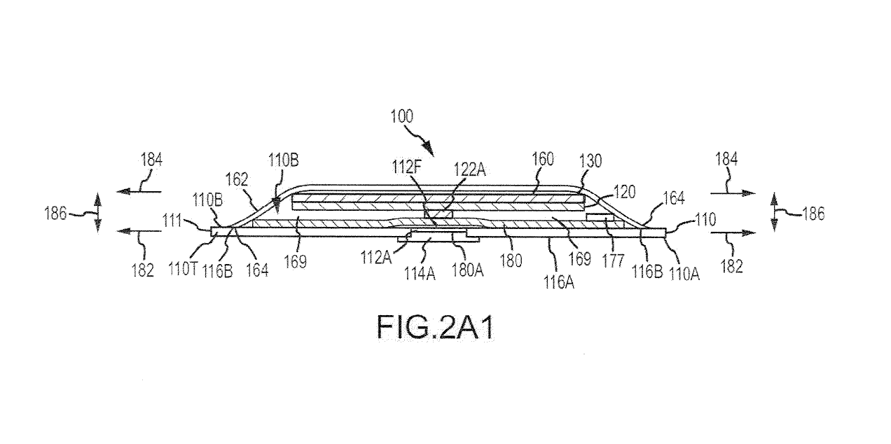

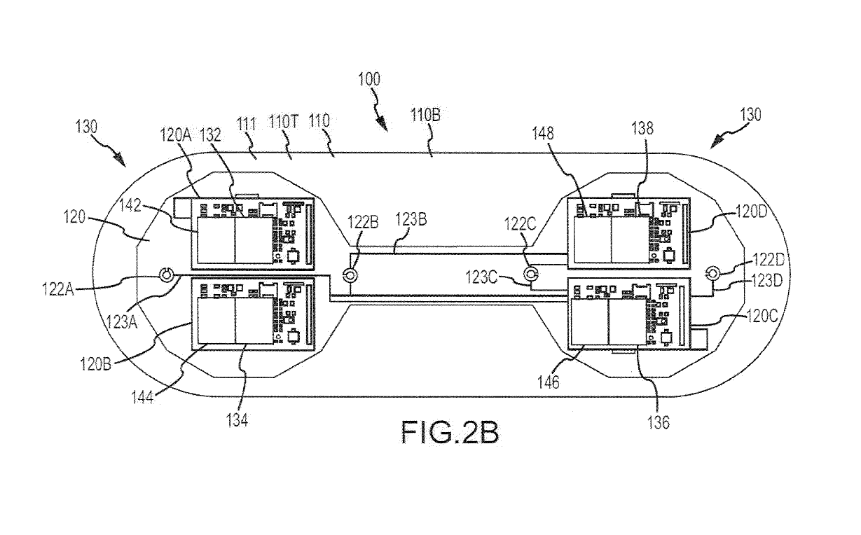

FIGS. 2A and 2A1 show a side cross-sectional view and an exploded view, respectively, of embodiments of the adherent device. The adherent device 100 may comprise an adherent patch 110 with an adhesive 116B, electrodes 112A, 112B, 112C, 112D with gels 114A, 114B, 114C, 114D, gel cover 180, temperature sensor 177, cover 162, and a printed circuit board (PCB) 120 with various circuitry for monitoring physiological sensors, communicating wirelessly with a remote center, and providing alerts when necessary. The adherent device 100 comprises at least two electrodes comprising two or more of electrodes 112A, 112B, 112C and 112D. Adherent device 100 may comprise a maximum dimension, for example a maximum length from about 4 to 10 inches, a maximum thickness along a profile of the device from about 0.2 inches to about 0.6 inches, and a maximum width from about 2 to about 4 inches.

The adherent patch 110 comprises a first side, or a lower side 110A, that is oriented toward the skin of the patient when placed on the patient. The adherent patch 110 may also comprise a tape 110T which is a material, preferably breathable, with an adhesive 116A to adhere to patient P. Electrodes 112A, 112B, 112C and 112D are affixed to adherent patch 110. In many embodiments, at least four electrodes are attached to the patch. Gels 114A, 114B, 114C and 114D can each be positioned over electrodes 112A, 112B, 112C and 112D, respectively, to provide electrical conductivity between the electrodes and the skin of the patient. Adherent patch 100 also comprises a second side, or upper side 110B. In many embodiments, electrodes 112A, 112B, 112C and 112D extend from lower side 110A through adherent patch 110 to upper side 110B. An adhesive 116B can be applied to upper side 110B to adhere structures, for example a breathable cover, to the patch such that the patch can support the electronics and other structures when the patch is adhered to the patient.

In many embodiments, adherent patch 110 may comprise a layer of breathable tape 110T, for example a tricot-knit polyester fabric, to allow moisture vapor and air to circulate to and from the skin of the patient through the tape. In many embodiments, breathable tape 110T comprises a backing material, or backing 111, with an adhesive. In many embodiments, the backing is conformable and/or flexible, such that the device and/or patch do not become detached with body movement. In many embodiments, the adhesive patch may comprise from 1 to 2 pieces, for example 1 piece. In many embodiments, adherent patch 110 comprises pharmacological agents, such as at least one of beta blockers, ace inhibitors, diuretics, steroid for inflammation, antibiotic, antifungal agent, and cortisone steroid. Patch 110 may comprise many geometric shapes, for example at least one of oblong, oval, butterfly, dogbone, dumbbell, round, square with rounded corners, rectangular with rounded corners, or a polygon with rounded corners. In specific embodiments, a thickness of adherent patch 110 is within a range from about 0.001″ to about 0.020″, length of the patch is within a range from about 2″ to about 10″, and width of the patch is within a range from about 1″ to about 5″.

In many embodiments, the adherent device 100 comprises a temperature sensor 177 disposed over a peripheral portion of gel cover 180 to allow the temperature near the skin to be measured through the breathable tape and the gel cover. Temperature sensor 177 can be affixed to gel cover 180 such that the temperature sensor can move when the gel cover stretches and tape stretch with the skin of the patient. Temperature sensor 177 may be coupled to temperature sensor circuitry 144 through a flex connection comprising at least one of wires, shielded wires, non-shielded wires, a flex circuit, or a flex PCB. The temperature sensor can be affixed to the breathable tape, for example through a cutout in the gel cover with the temperature sensor positioned away from the gel pads. A heat flux sensor can be positioned near the temperature sensor for example to measure heat flux through to the gel cover.

The adherent device comprises electrodes 112A, 112B, 112C and 112D configured to couple to tissue through apertures in the breathable tape 110T. Electrodes 112A, 112B, 112C and 112D can be fabricated in many ways, for example printed on a flexible connector 112F, such as silver ink on polyurethane. In some embodiments, the electrodes may comprise at least one of carbon-filled ABS plastic, Ag/AgCl, silver, nickel, or electrically conductive acrylic tape. The electrodes may comprise many geometric shapes to contact the skin, for example at least one of square, circular, oblong, star shaped, polygon shaped, or round. In specific embodiments, a dimension across a width of each electrode is within a range from about 002″ to about 0.050″. In specific embodiments, the two inside electrodes may comprise force, or current electrodes, with a center to center spacing within a range from about 20 to about 50 mm. In specific embodiments, the two outside electrodes may comprise measurement electrodes, for example voltage electrodes, and a center-center spacing between adjacent voltage and current electrodes is within a range from about 15 mm to about 35 mm. Therefore, in many embodiments, a spacing between inner electrodes may be greater than a spacing between an inner electrode and an outer electrode.

In many embodiments, gel 114A, or gel layer, comprises a hydrogel that is positioned on electrode 112A and provides a conductive interface between skin and electrode, so as to reduce impedance between electrode/skin interface. The gel may comprise water, glycerol, and electrolytes, pharmacological agents, such as beta blockers, ace inhibitors, diuretics, steroid for inflammation, antibiotic, and antifungal agents. Gels 114A, 114B, 114C and 114D can be positioned over electrodes 112A, 112B, 112C and 112D, respectively, so as to couple electrodes to the skin of the patient. The flexible connector 112F comprising the electrodes can extend from under the gel cover to the PCB to connect to the PCB and/or components supported thereon. For example, flexible connector 112F may comprise flexible connector 122A to provide strain relief.

A gel cover 180, or gel cover layer, for example a polyurethane non-woven tape, can be positioned over patch 110 comprising the breathable tape to inhibit flow of gels 114A-114D through breathable tape 110T. Gel cover 180 may comprise at least one of a polyurethane, polyethylene, polyolefin, rayon, PVC, silicone, non-woven material, foam, or a film. Gel cover 180 may comprise an adhesive, for example an acrylate pressure sensitive adhesive, to adhere the gel cover to adherent patch 110. In many embodiments, the gel cover can regulate moisture of the gel near the electrodes so as to keeps excessive moisture, for example from a patient shower, from penetrating gels near the electrodes. A PCB layer, for example the flex PCB 120, or flex PCB layer, can be positioned over gel cover 180 with electronic components 130 connected and/or mounted to the flex PCB 120, for example mounted on flex PCB so as to comprise an electronics layer disposed on the flex PCB layer. In many embodiments, the gel cover may avoid release of excessive moisture form the gel, for example toward the electronics and/or PCB modules. In many embodiments, a thickness of gel cover is within a range from about 0.0005″ to about 0.020″. In many embodiments, gel cover 180 can extend outward from about 0-20 mm from an edge of gels. Gel layer 180 and breathable tape 110T comprise apertures 180A, 180B, 180C and 180D through which electrodes 112A-112D are exposed to gels 114A-114D.

In many embodiments, device 100 includes a printed circuitry, for example a PCB module that includes at least one PCB with electronics component mounted thereon. The printed circuit may comprise polyester film with silver traces printed thereon. Rigid PCB's 120A, 120B, 120C and 120D with electronic components may be mounted on the flex PCB 120. In many embodiments, the PCB module comprises two rigid PCB modules with associated components mounted therein, and the two rigid PCB modules are connected by flex circuit, for example a flex PCB. In specific embodiments, the PCB module comprises a known rigid FR4 type PCB and a flex PCB comprising known polyimide type PCB. Batteries 150 may be positioned over the flex PCB and electronic components. Batteries 150 may comprise rechargeable batteries that can be removed and/or recharged. A cover 162 may be placed over the batteries, electronic components and flex PCB. In specific embodiments, the PCB module comprises a rigid PCB with flex interconnects to allow the device to flex with patient movement. The geometry of flex PCB module may comprise many shapes, for example at least one of oblong, oval, butterfly, dogbone, dumbbell, round, square, rectangular with rounded corners, or polygon with rounded corners. In specific embodiments the geometric shape of the flex PCB module comprises at least one of dogbone or dumbbell. The PCB module may comprise a PCB layer with flex PCB 120 that can be positioned over gel cover 180 and electronic components 130 connected and/or mounted to flex PCB 120. In many embodiments, the adherent device may comprise a segmented inner component, for example the PCB, for limited flexibility.

In many embodiments, an electronics housing 160 encapsulates the electronics layer. Electronics housing 160 may comprise an encapsulant, such as a dip coating, which may comprise a waterproof material, for example silicone, epoxy, other adhesives and/or sealants. In many embodiments, the PCB encapsulant protects the PCB and/or electronic components from moisture and/or mechanical forces. The encapsulant may comprise silicone, epoxy, other adhesives and/or sealants. In some embodiments, the electronics housing may comprising metal and/or plastic housing and potted with aforementioned sealants and/or adhesives.

In many embodiments, cover 162 can encase the flex PCB, electronics, and/or adherent patch 110 so as to protect at least the electronics components and the PCB. In some embodiments, cover 162 can be adhered to adherent patch 110 with an adhesive 164 or adhesive 116B on an underside of cover 162. In many embodiments, cover 162 attaches to adherent patch 110 with adhesive 116B, and cover 162 is adhered to the PCB module with an adhesive 161 on the upper surface of the electronics housing. Cover 162 can comprise many known biocompatible cover materials, for example silicone, an outer polymer cover to provide smooth contour without limiting flexibility, a breathable fabric, or a breathable water resistant cover. In some embodiments, the breathable fabric may comprise polyester, nylon, polyamide, and/or elastane (Spandex™). Work in relation to embodiments of the present invention suggests that these coatings can be important to keep excessive moisture from the gels near the electrodes and to remove moisture from body so as to provide patient comfort.

In many embodiments, cover 162 can be attached to adherent patch 110 with adhesive 116B such that cover 162 stretches and/or retracts when adherent patch 110 stretches and/or retracts with the skin of the patient. For example, cover 162 and adherent patch 110 can stretch in two dimensions along the length and width of the adherent patch with the skin of the patient, and stretching along the length can increase spacing between electrodes. Stretching of the cover and adherent patch 110 can extend the time the patch is adhered to the skin as the patch can move with the skin. Electronics housing 160 can be smooth and allow breathable cover 162 to slide over electronics housing 160, such that motion and/or stretching of cover 162 is slidably coupled with housing 160. The PCB can be slidably coupled with adherent patch 110 that comprises breathable tape 110T, such that the breathable tape can stretch with the skin of the patient when the breathable tape is adhered to the skin of the patient, for example along two dimensions comprising the length and the width.

The breathable cover 162 and adherent patch 110 comprise breathable tape that can be configured to couple continuously for at least one week the at least one electrode to the skin so as to measure breathing of the patient. The breathable tape may comprise the stretchable breathable material with the adhesive and the breathable cover may comprises a stretchable breathable material connected to the breathable tape, as described above, such that both the adherent patch and cover can stretch with the skin of the patient. Arrows 182 show stretching of adherent patch 110, and the stretching of adherent patch can be at least two dimensional along the surface of the skin of the patient. As noted above, connectors 122A-122D between PCB 130 and electrodes 112A-112D may comprise insulated wires that provide strain relief between the PCB and the electrodes, such that the electrodes can move with the adherent patch as the adherent patch comprising breathable tape stretches. Arrows 184 show stretching of cover 162, and the stretching of the cover can be at least two dimensional along the surface of the skin of the patient.

The PCB 120 may be adhered to the adherent patch 110 comprising breathable tape 110T at a central portion, for example a single central location, such that adherent patch 110 can stretched around this central region. The central portion can be sized such that the adherence of the PCB to the breathable tape does not have a substantial effect of the modulus of the composite modulus for the fabric cover, breathable tape and gel cover, as described above. For example, the central portion adhered to the patch may be less than about 100 mm², for example with dimensions that comprise no more than about 10% of the area of patch 110, such that patch 110 can stretch with the skin of the patient. Electronics components 130, PCB 120, and electronics housing 160 are coupled together and disposed between the stretchable breathable material of adherent patch 110 and the stretchable breathable material of cover 160 so as to allow the adherent patch 110 and cover 160 to stretch together while electronics components 130, PCB 120, and electronics housing 160 do not stretch substantially, if at all. This decoupling of electronics housing 160, PCB 120 and electronic components 130 can allow the adherent patch 110 comprising breathable tape to move with the skin of the patient, such that the adherent patch can remain adhered to the skin for an extended time of at least one week.

An air gap 169 may extend from adherent patch 110 to the electronics module and/or PCB, so as to provide patient comfort. Air gap 169 allows adherent patch 110 and breathable tape 110T to remain supple and move, for example bend, with the skin of the patient with minimal flexing and/or bending of PCB 120 and electronic components 130, as indicated by arrows 186. PCB 120 and electronics components 130 that are separated from the breathable tape 110T with air gap 169 can allow the skin to release moisture as water vapor through the breathable tape, gel cover, and breathable cover. This release of moisture from the skin through the air gap can minimize, and even avoid, excess moisture, for example when the patient sweats and/or showers. Gap 169 extends from adherent patch 110 to the electronics module and/or PCB a distance within a range from about 0.25 mm to about 4 mm.

In many embodiments, the adherent device comprises a patch component and at least one electronics module. The patch component may comprise adherent patch 110 comprising the breathable tape with adhesive coating 116A, at least one electrode, for example electrode 112A and gel 114A. The at least one electronics module can be separable from the patch component. In many embodiments, the at least one electronics module comprises the flex PCB 120, electronic components 130, electronics housing 160 and cover 162, such that the flex PCB, electronic components, electronics housing and cover are reusable and/or removable for recharging and data transfer, for example as described above. In specific embodiments, the electronic module can be adhered to the patch component with a releasable connection, for example with Velcro™, a known hook and loop connection, and/or snap directly to the electrodes. Monitoring with multiple adherent patches for an extended period is described in U.S. Pub. No. 2009-0076345-A1, published on 3-19-2009, the full disclosure of which has been previously incorporated herein by reference, and which adherent patches and methods are suitable for combination in accordance with embodiments described herein.

The adherent device 100, shown in FIG. 2A, may comprise an X-axis, Y-axis and Z-axis for use in determining the orientation of the adherent device 100 and/or the patient P. Electric components 130 may comprise a 3D accelerometer. As the accelerometer of adherent device 100 can be sensitive to gravity, inclination of the patch relative to an axis of the patient can be measured, for example when the patient stands. Vectors from a 3D accelerometer can be used to determine the orientation of a measurement axis of the patch adhered on the patient and can be used to determine the angle of the patient, for example whether the patient is laying horizontally or standing upright, when measured relative to the X-axis, Y-axis and/or X-axis of adherent device 100.

FIG. 2B shows a PCB and electronic components over adherent patch 110. In some embodiments, PCB 120, for example a flex PCB, may be connected to electrodes 112A, 112B, 112C and 112D of FIG. 2A with connectors 122A, 122B, 122C and 122D, respectively, and may include traces 123A, 123B, 123C and 123D that extend to connectors 122A, 122B, 122C and 122D. In some embodiments, connectors 122A-122D may comprise insulated wires and/or a film with conductive ink that provide strain relief between the PCB and the electrodes. Examples of structures to provide strain relief are also described in U.S. Pub. No. 2009-0076345-A1, entitled “Adherent Device with Multiple Physiological Sensors”, filed on Sep. 12, 2008 as noted above.

Electronic components 130 comprise components to take physiologic measurements, transmit data to remote center 106 and receive commands from remote center 106. In many embodiments, electronics components 130 may comprise known low power circuitry, for example complementary metal oxide semiconductor (CMOS) circuitry components. Electronics components 130 comprise a temperature sensor, an activity sensor and activity circuitry 134, impedance circuitry 136 and electrocardiogram circuitry, for example ECG circuitry 138. In some embodiments, electronic circuitry 130 may comprise a microphone and microphone circuitry 142 to detect an audio signal, such as heart or respiratory sound, from within the patient.

Electronic circuitry 130 may comprise a temperature sensor, for example a thermistor in contact with the skin of the patient, and temperature sensor circuitry 144 to measure a temperature of the patient, for example a temperature of the skin of the patient. A temperature sensor may be used to determine the sleep and wake state of the patient, which may decrease during sleep and increase during waking hours. Work in relation to embodiments of the present invention suggests that skin temperature may affect impedance and/or hydration measurements, and that skin temperature measurements may be used to correct impedance and/or hydration measurements. In some embodiments, increase in skin temperature or heat flux can be associated with increased vaso-dilation near the skin surface, such that measured impedance measurement decreased, even though the hydration of the patient in deeper tissues under the skin remains substantially unchanged. Thus, use of the temperature sensor can allow for correction of the hydration signals to more accurately assess the hydration, for example extra cellular hydration, of deeper tissues of the patient, for example deeper tissues in the thorax.

Activity sensor and activity circuitry 134 can comprise many known activity sensors and circuitry. In many embodiments, the accelerometer comprises at least one of a piezoelectric accelerometer, capacitive accelerometer or electromechanical accelerometer. The accelerometer can comprise a 3-axis accelerometer to measure at least one of an inclination, a position, an orientation or acceleration of the patient in three dimensions. Work in relation to embodiments of the present invention suggests that three dimensional orientation of the patient and associated positions, for example sitting, standing, lying down, can be very useful when combined with data from other sensors, for example hydration data.

Impedance circuitry 136 can generate both hydration data and respiration data. In many embodiments, impedance circuitry 136 is electrically connected to electrodes 112A, 112B, 112C and 112D of FIG. 2A in a four pole configuration, such that electrodes 112A and 112D comprise outer electrodes that are driven with a current and comprise force electrodes that force the current through the tissue. The current delivered between electrodes 112A and 112D generates a measurable voltage between electrodes 112B and 112C, such that electrodes 112B and 112C comprise inner, sense, electrodes that sense and/or measure the voltage in response to the current from the force electrodes. In some embodiments, electrodes 112B and 112C may comprise force electrodes and electrodes 112A and 112D may comprise sense electrodes. The voltage measured by the sense electrodes can be used to measure the impedance of the patient and determine the respiration rate and/or hydration of the patient. The electrocardiogram circuitry may be coupled to the sense electrodes to measure the electrocardiogram signal, for example as described in U.S. Pub. No. 2009-0076345-A1, entitled “Adherent Device with Multiple Physiological Sensors”, published on Mar. 29, 2009, previously incorporated by reference and suitable for combination in accordance with embodiments described herein. In many embodiments, impedance circuitry 136 can be configured to determine respiration of the patient. In specific embodiments, the impedance circuitry can measure the hydration at 25 Hz intervals, for example at 25 Hz intervals using impedance measurements with a frequency from about 0.5 kHz to about 20 kHz.

ECG circuitry 138 can generate electrocardiogram signals and data from two or more of electrodes 112A, 112B, 112C and 112D in many ways. In some embodiments, ECG circuitry 138 is connected to inner electrodes 112B and 122C, which may comprise sense electrodes of the impedance circuitry as described above. In many embodiments, the ECG circuitry may measure the ECG signal from electrodes 112A and 112D when current is not passed through electrodes 112A and 112D.

Electronic circuitry 130 may comprise a processor 146 that can be configured to control a collection and transmission of data from the impedance circuitry electrocardiogram circuitry and the accelerometer. Processor 146 comprises a tangible medium, for example read only memory (ROM), electrically erasable programmable read only memory (EEPROM) and/or random access memory (RAM). Electronic circuitry 130 may comprise real time clock and frequency generator circuitry 148. In some embodiments, processor 146 may comprise the frequency generator and real time clock. In many embodiments, device 100 comprises a distributed processor system, for example with multiple processors on device 100.

In many embodiments, electronics components 130 comprise wireless communications circuitry 132 to communicate with remote center 106. PCB 120 may comprise an antenna to facilitate wireless communication. The antenna may be integral with PCB 120 or may be separately coupled thereto. The wireless communication circuitry can be coupled to the impedance circuitry, the electrocardiogram circuitry and the accelerometer to transmit to a remote center with a communication protocol at least one of the hydration signal, the electrocardiogram signal or the inclination signal. In specific embodiments, wireless communication circuitry 132 is configured to transmit the hydration signal, the electrocardiogram signal and the inclination signal to the remote center either directly or through gateway 102. The communication protocol comprises at least one of Bluetooth, ZigBee, WiFi, WiMAX, IR, amplitude modulation or frequency modulation. In many embodiments, the communications protocol comprises a two way protocol such that the remote center is capable of issuing commands to control data collection.

In many embodiments, the electrodes are connected to the PCB with a flex connection, for example trace 123A, 123B, 123C and 123D of flex PCB 120, so as to provide strain relief between the electrodes 112A, 112B, 112C and 112D and the PCB. In such embodiments, motion of the electrodes relative to the electronics modules, for example rigid PCB's 120A, 120B, 120C and 120D with the electronic components mounted thereon, does not compromise integrity of the electrode/hydrogel/skin contact. In many embodiments, the flex connection comprises at least one of wires, shielded wires, non-shielded wires, a flex circuit, or a flex PCB. In specific embodiments, the flex connection may comprise insulated, non-shielded wires with loops to allow independent motion of the PCB module relative to the electrodes.

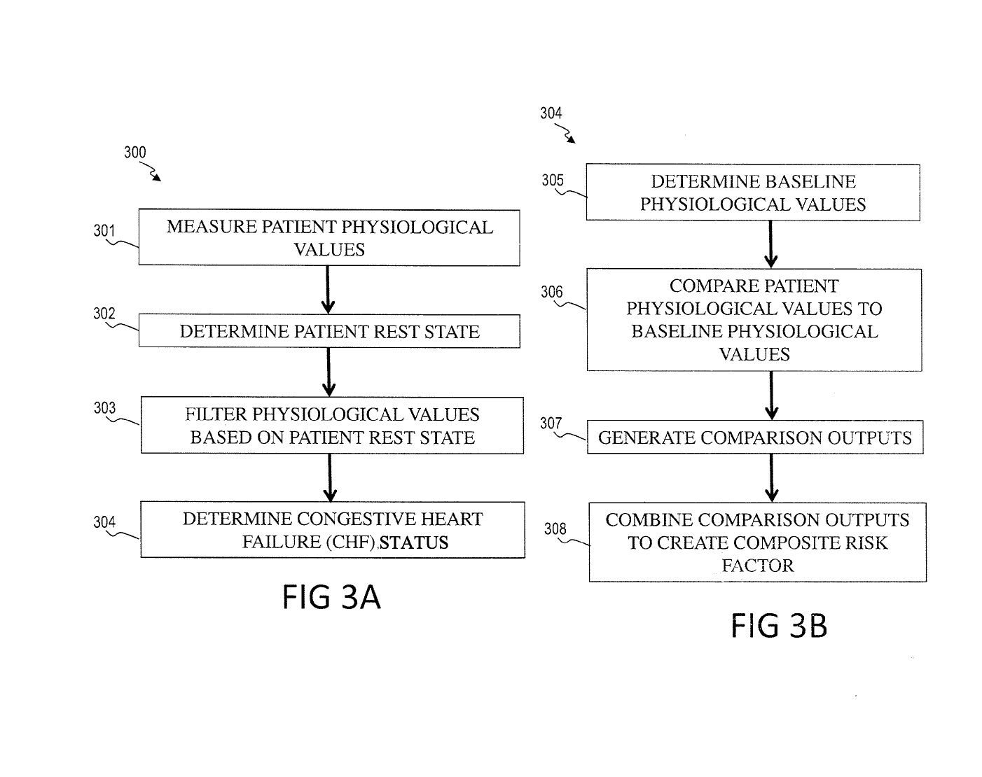

FIG. 3A shows a method of determining patient CHF status 300 comprising measuring patient physiological values 301, determining a patient rest state 302, filtering one or more physiological values based on a patient rest state 303, and determining CHF status 304. Measuring or determining one or more physiological values 301 can comprise one or more of generating bioimpedence measurements, ECG recordings, and 3-axis accelerometer measurements. Generating bioimpedance measurements may include measuring resistance, reactance or changes in resistance and/or reactance over time, for example. At least one of a fluid bioimpedance contribution, fat bioimpedance contribution or ion bioimpedance contribution may be combined to generate one or more physiological values. Frequency sweeps may be utilized to quantify the contribution of non-fluid parameters, such as fat, ions or other components, to derive fluid-specific values for long-term tracking. The method allows for an identification of whether a signal and result are reliable, based on the identification of other components. For example, a high ion measurement may indicate the need to re-measure a patient's fluid status. Spot recording of physiological components may be accomplished by adapting a stimulation waveform to obtain a fluid bioimpedance value. Adapting may include changing a signal's amplitude, frequency or shape, for example. Adapting may reduce the number of false positives, for example. Bioimpedance measurements can be used to determine a breath parameter such as breath volume, breath rate, or breathing effort. The breath parameter values may be further filtered before use. For example, in some embodiments, the breath parameter values are low-pass filtered before use, using an averaging finite impulse response filter of length 20.

ECG recordings can be used to determine heart rate and heart rhythm. For example, ECG circuitry to generate electrocardiogram signals and data from two or more electrodes. A PiiX device can monitor real-time ECG and transmit a continuous heart rate stream as well as arrhythmia detections and heart rate strips. Heart rate values can be more specifically defined to include only non-noisy, non-premature, non-outlier beats. Arrhythmias such as atrial fibrillation (AF) and premature ventricular complexes (PVCs) are associated with higher CHF risk. Devices such as the PiiX have on-board algorithms for arrhythmia detection, which can monitor arrhythmias in real-time. Heart rate strips, such as those created by a PiiX device, can be manually read by ECG technicians to monitor and determine CHF status. A measure of AF burden is a measure of CHF status. If the AF detection values from a PiiX device are directly used, the derived AF burden numbers can be scaled to account for AF detection algorithm sensitivity and specificity. An alternative is to use the manual over-read values as generated by ECG technicians for CHF risk determination.

3-axis accelerometer measurements can be used to measure activity intensity (ActivIntens) in milliGs (mG) over time in addition to patient posture. Activity sensors and activity circuitry can comprise many known activity sensors and circuitry. In many embodiments, the accelerometer comprises at least one of a piezoelectric accelerometer, capacitive accelerometer or electromechanical accelerometer. The accelerometer can comprise a 3-axis accelerometer to measure at least one of an inclination, a position, an orientation or acceleration of the patient in three dimensions. 3-axis accelerometer measurements can be generated at regular intervals, for example every 4 seconds.

Work in relation to embodiments of the present invention suggests that three dimensional orientation of the patient and associated positions, for example sitting, standing, lying down, can be very useful when combined with data from other sensors, for example ECG data and breath parameter data. As the accelerometer can be sensitive to gravity, inclination of the patch relative to an axis of the patient can be measured, for example when the patient stands. Vectors from a 3D accelerometer can be used to determine the orientation of a measurement axis of the patient and can be used to determine the angle of the patient, for example whether the patient is laying horizontally or standing upright, when measured relative to the X-axis, Y-axis and/or Z-axis.

To ensure accurate determinations of whether the patient is standing upright or lying down, 3-D measurements provided by the accelerometer may be initialized to determine whether offsets should be applied to correctly ascertain patient posture. For example, in one embodiment an upright posture offset is calculated based on an assumption that patients are upright when active—wherein upright posture is represented by a Z-axis angle of 90° relative to the horizon. During a period of patient activity—at which time it can be assumed the patient is upright and therefore oriented at approximately 90° relative to the horizon—3D orientation data is collected from the 3D accelerometer. An average posture of the patient is compared to the expected upright posture value (e.g., z-axis angle of 90°) and an offset is calculated to correct for the difference between the measured posture and expected posture. For example, if the average measured upright posture of the patient is measured to be 60° relative to the horizon, this suggests that an upright posture offset of approximately 30° should be utilized to correctly estimate patient posture. Subsequent determinations of the posture of the patient take into account this initial upright posture offset value in order to correctly determine patient posture.

Measuring patient physiological values 301 may include applying a noise filter to measured physiological values. For example, 3-axis accelerometer measurements may include a high-frequency component and a low frequency component, wherein high-frequency components typically correspond to physiological values such as activity intensity and activity duration, and low-frequency components typically correspond to physiological values such as body posture. High or low frequency components of 3-axis accelerometer measurements may be filtered out depending on which physiological value is being measured.

In measuring patient activity, 3-axis accelerometer measurements can be subjected to a high-pass filter to remove low-frequency components. Activity may be measured in a number of ways. For example, {ActivIntens(i)} can denote the set of activity intensity samples in a moving window, w. The moving window can be of a length, for example 12 hours, to monitor diurnal activity changes per, for example, 24 hours to track daily activity changes. Longer durations may be monitored to track long-term activity trends. An activity flag (ActivFlag) can be used to indicate when activity intensity exceeds a threshold (ActivThreshold), e.g., 105mG, which can correspond to a walking-level activity. For example, for {ActivFlag(i)}={ActivIntens(i)>=ActivThreshold}; the activity flag ActivFlag is true when the activity intensity exceeds 105 mG, or any other suitable inputted threshold.

Other activity values may also be measured for a given time window, for example activity duration=Σ ActivFlag(i), peak activity=max({ActivIntens(i)}), mean activity intensity=average({ActivIntens(i)}). These values may be used to track worsening CHF risk in a patient.

Noise filters may also be applied to physiological values, such as bioimpedance measurements and ECG recordings. The type of noise filter applied can depend on the specific physiological value. Bioimpedance measurements can be filtered by considering the median of a set of measurements to avoid bias from outliers. Erroneous or unreliable bioimpedance measurements, for example those caused by improper patch adhesion, can be filtered by discarding measurements above or below thresholds or measurements outside a certain range, e.g., 5 ohms<and<120 ohms, or by discarding measurements corresponding to a durations of high variability. Two or more of each filtering methods can also be combined.

Breath rates based on bioimpedance measurements may be further or alternatively filtered by a number of filtering methods, including high-pass filters to remove low-frequency artifacts, baseline removal to remove linear and DC values over the signal measurement duration, baseline corrections to remove any transient low-frequency changes, median filters to remove high-frequency artifacts, and band-pass filters. Two or more of each filtering methods can also be combined. The same methods, excluding baseline correction, can be used to filter breath volume based on bioimpedance measurements.

ECG measurements used to detect heart rates and arrhythmias can be filtered using low and high-pass frequency ECG sub-bands to detect and discard noise durations wherein measurements are not considered reliable. High-frequency ECG sub-bands can be used to detect noise such as muscle artifacts and electron motion artifacts, and low-frequency ECG sub-bands can be used to detect noise such as signal loss and baseline drift artifacts. Heart rate streams can be filtered to include only non-noisy beats, non-premature beats, non-outlier beats, or a combination thereof. ECG measurements may also be filtered manually. For example, ECG strips captured from a device can be examined by experts to identify artifacts and other noise to identify unreliable durations.

Determining a patient rest state 302 may be determined based on activity level, activity level and posture, and the time of day. For example, a patient can be determined to be at rest if activity level is below a threshold, for example 10 mG. A patient can be determined to be at rest if activity level is below a threshold, for example 10 mG, and if body angle is below a threshold, for example 10 degrees. A patient can be determined to be at rest based on time of day, for example midnight to 4:00 o'clock am. Examples of rest states may include at rest, or not at rest. A rest period or rest instance is a period or instance in which a patient is at rest. At rest can comprise sleep and sedentary activity. For example, a period in which a patient is in a seated position can be a rest period.

Three-axis accelerometer measurements can be used to measure and determine patient posture, such as body angle during activity or whether a patient is supine. The low-pass filtered values are used to compute the posture-related offset trends and patient's body posture angle over time. A measure for whether a patient is supine is based mainly on the z-axis measurements. For example, if the distribution of z-axis measurements at rest is between an intermediate range, e.g., −600 mG to 600 mG, then the patient is deemed to be non-supine at rest, else the patient is considered to be supine at rest.

An additional indicator of supine patient posture compares the z-axis measurements and the x-axis and y-axis measurements during rest. For example, if the magnitude of the z-axis measurement is the highest among the 3-axis measurements and/or the magnitude of the z-axis measurement is greater than the sum of squares of the x-axis and y-axis measurements, then the patient is deemed to be supine at rest. In another example, if the magnitude of the y-axis measurement is the highest among the 3-axis measurements and/or the magnitude of the y-axis measurement is greater than the sum of squares of the x-axis and z-axis measurements, the patient is deemed be at lying on their side at rest.

The time of day, a patient's sleep schedule as determined by a physician or medical professional, and sleep schedule data provided by the patient, for example via phone or web portal, can also be used to determine whether a patient is at rest. However, use of these metrics may preclude real-time determination of rest instances.

Methods of filtering physiological values based on patient rest state 303 depend on the physiological value being filtered. Physiological values such as heart rate, breath rate, and breath volume can be unreliable metrics for monitoring and determining CHF risk if measured while a patient is not at rest. The methods provided herein further comprise filtering one or more physiological values, such as heart rate, breath rate, breath volume, and body posture, based on whether the patient is at rest. Filtering can comprise rejecting or disregarding physiological values which are not measured at rest. A filtered value is one which is not rejected or disregarded during filtering. Some physiological values are valuable CHF risk metrics whether or not the patient is at rest. Thus, filtering can be physiological value-specific and may not discard any of a certain physiological value based on a rest state. For example, in some embodiments, no fluid status or arrhythmia values or measurements are discarded during filtering, regardless of a patient rest state.

In other embodiments, one or more of heart rate, breath rate, and breath volume can be filtered to disregard any value measured while a patient is not at rest. Body posture at rest and not at rest can each be used to determining worsening or improving CHF risk. Therefore in some examples, each of body posture measured at rest and not at rest may be filtered. For example, a decrease in supine body posture at rest may indicate worsening CHF risk. In this example, body posture measurements are filtered to include only those measured while a patient is in a rest state. In another example, a decrease in upright or erect body posture during patient activity or non-rest periods may indicate worsening CHF risk. In this example, body measurements are filtered to include only those measured while a patient is active, or in a non-resting state.

Patient activity intensity and duration can be used to indicate worsening or improving CHF risk. These physiological values may be filtered to include only those measured while a patient is active, or in a non-resting state. In another example, these physiological values may be filtered to include only those measured while a patient is in a rest state in order to determine an amount of movement or tossing and turning during a given rest period.

FIG. 3B shows a method of determining patient CHF status 304 comprising determining baseline physiological values 305, comparing patient physiological values to baseline physiological values 306, generating comparison outputs 307, and combining comparison outputs to create composite risk factors 308. Determining baseline physiological values 305 includes considering, such as combining, one or more of pre-test likelihood of hospitalization metrics, patient demographics, physiological values determined during a patient hospitalization, historic physiological values determined after a patient hospitalization, clinical lab results, and other suitable metrics. Physiological values can be filtered, unfiltered, or a combination thereof. Pre-test likelihood of hospitalization metrics can include family medical history of a patient, patient medical history, patient medical treatment plan, and patient drug regimen. Patient demographics can include one or more of age, ethnicity, and gender.

Baseline values may be set based on only one physiological value, such as one measured during an in-hospital phase, or based on a plurality of values. For example, patient posture during rest instances measured during hospitalization may be used as a baseline value for patient posture. Baseline values can additionally or alternatively be set based on a threshold value appropriate for a patient demographic or a particular patient's relevant pre-test likelihood of hospitalization metrics. For example, a baseline or threshold heart rate value may be adjusted based on the age of a patient. In another embodiment, baseline values may be defined or modified based on values collected from the patient while healthy. For example, a set of baseline values may be based on values collected from the patient at one or more annual exams, and may represent a “healthy” baseline status of the patient.

Comparing patient physiological values to baseline physiological values 306 in some examples allows for sequential diagnoses from data gathered by, for example, a PiiX device to be more accurately tuned to a specific patient or demographic of patient to generate a post-test likelihood of hospitalization due to, for example, CHF.

Comparisons of patient physiological values to baseline values may be quantified or illustrated by generating comparison outputs 307. Comparison outputs may be relative percentage differences between physiological values and baseline values, quantitative differences between physiological values and baseline values, or flags which indicate if a physiological value has met a comparison condition. For example, a flag may be generated if a physiological value exceeds a baseline value (relative change) or threshold value (absolute change).

In measuring and monitoring patient activity, baseline values may be previously measured activity values from within a given time window. For example, a decrease accumulator can be used to monitor monotonic decreases in mean activity intensity over a given number, N, windows and alert at an accumulation threshold, or generally indicate worsening CHF status in a patient. For example, if mean activity intensity at window w+1<=(1+Δ)*mean activity intensity at window w, then accumulator at window w+1=1+accumulator at window w, else ( ).

A fall in peak activity intensity below a fraction of the patient's own healthy peak activity level can indicate worsening CHF status in a patient. For example, a 50% decrease in the peak activity intensity compared to peak activity intensity at normal health, or at a value set by a physician or medical professional.

A fall in peak activity intensity below a fraction of the age and gender adjusted peak activity level can indicate worsening CHF status in a patient. For example, a 50% decrease in the peak activity intensity compared to a peak activity level threshold for an age group, a gender, an age and gender group, or a value set by a physician or medical professional.

Additionally, the 3-axis accelerometer can measure body posture, which may indicate a worsening CHF status in a patient. For example, a decreasing trend in the body posture or supine index or a consistently high body posture or supine index for an extended duration, e.g., 2 days, evidences patient's inability to lay down during rest and indicates a worsening CHF status. Similarly, a slumped or non-erect body posture during active instances, or an increased trend in the same, can indicate a worsening CHF status.

Breath rate values can be used to monitor and determine patient CHF. For example, a patient breath rate in excess of a threshold (e.g., 20 breaths per minute) while at rest can indicate worsening CHF status. A trend in increased patient breath rate while at rest can indicate worsening CHF status. A patient breath rate below a threshold (e.g., 10 breaths per minute) can indicate an improving CHF status. A trend in decreased patient breath rate while at rest can indicate an improving CHF status. Filtered breath rate values may be used to monitor and determine CHF status of a patient in real-time.

Filtered heart rate values can be used to monitor and determine patient CHF status. For example, {HR(i)} can denote the set of heart rate values at rest in a moving window w. The moving window can be, for example, 12 hours long to monitor diurnal activity changes per 24 hours to track daily activity changes. Longer durations may be monitored to track long-term activity trends. One or more flags may be asserted for each moving window. If a given flag or multiple flags are continually asserted over N windows (e.g., 4 windows or 2 days), or is asserted in N out of the last M windows (e.g., 12 hours out of the last 16 hours), then CHF risk increases.

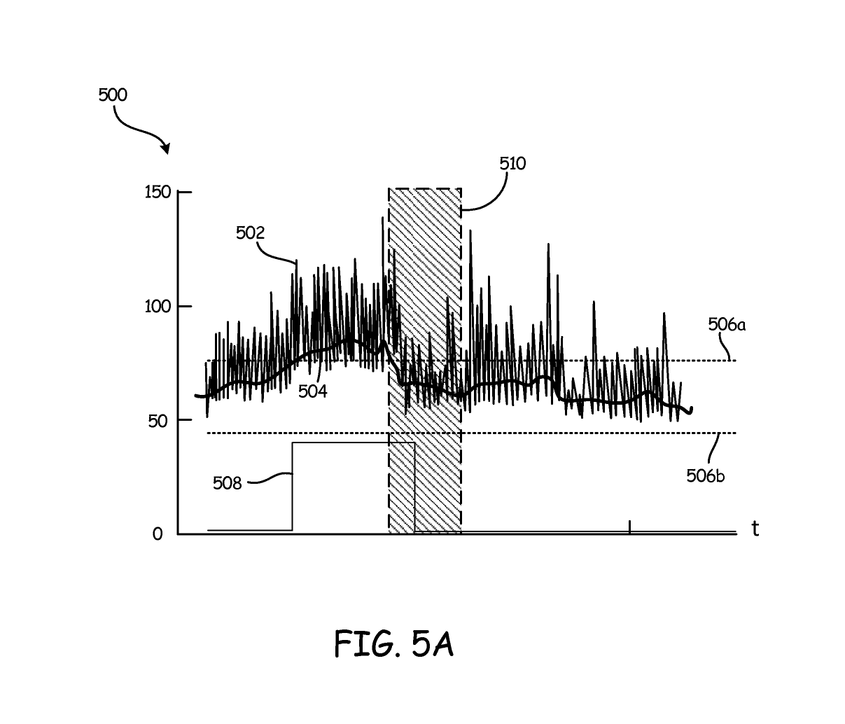

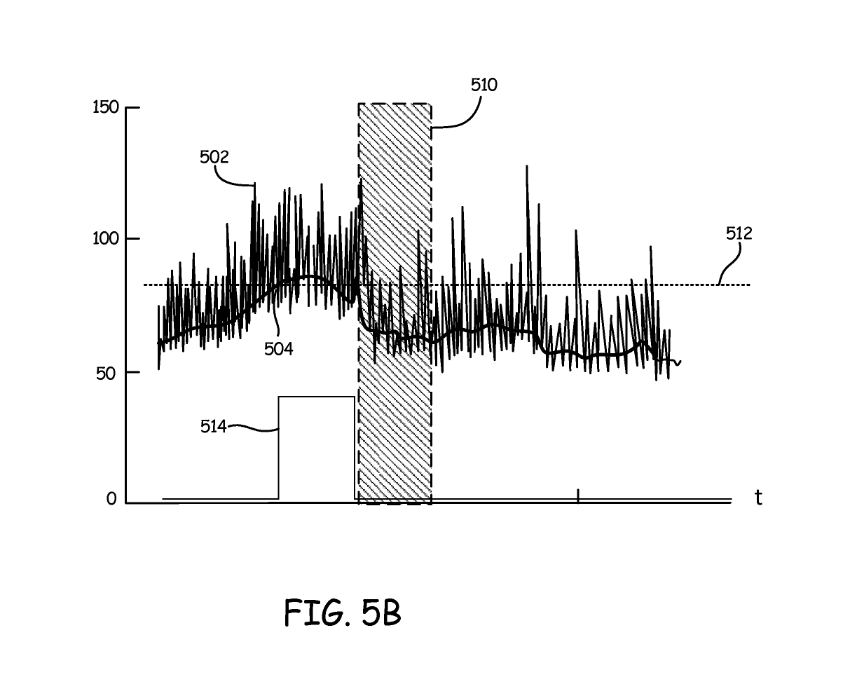

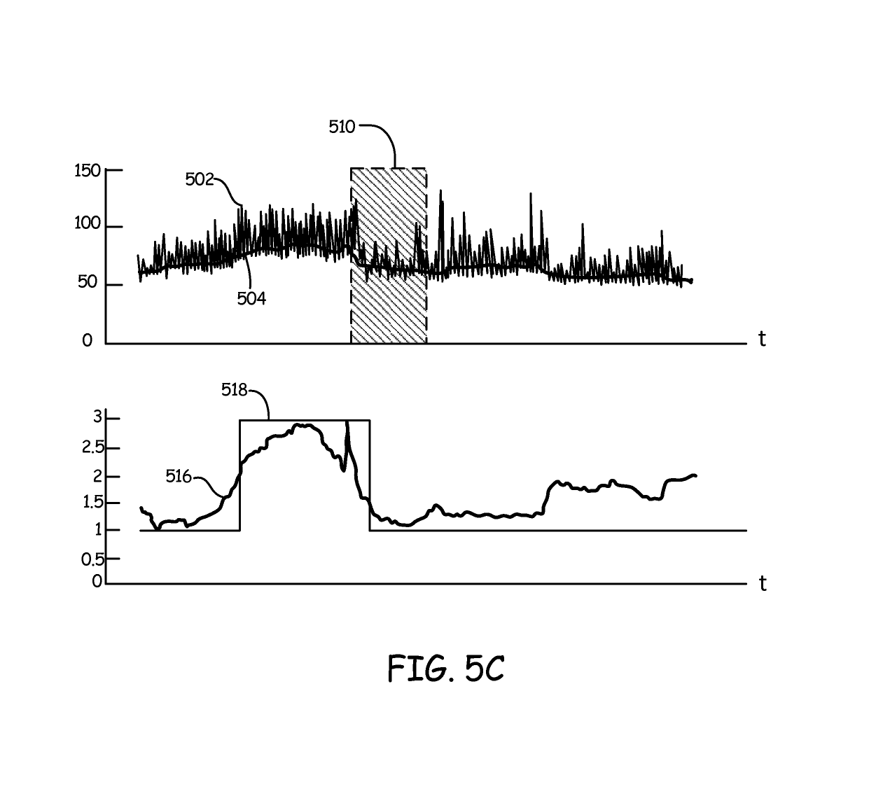

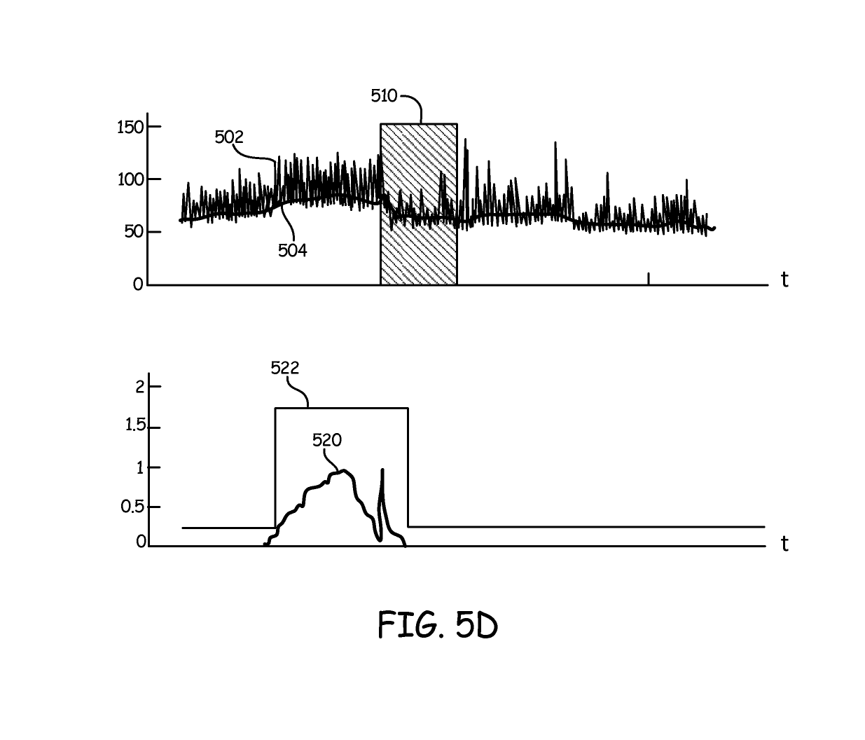

FIGS. 5A-5D are charts illustrating analysis of filtered heart rate data in a moving window according to various techniques of this disclosure.

In the example illustrated in FIG. 5A, chart 500 illustrates filtered heart rate data 502 (e.g., heart rate at rest data), median or outlier-removed heart rate data 504, and max-min heart rate thresholds 506a and 506b, respectively. In the embodiment illustrated in FIG. 5A, an alert illustrated by line 508 is triggered if outlier-removed heart rate data 504 exceeds either of the max-min heart rate thresholds 506a, 506b. For example, maximum heart rate threshold 506a may be set at 75 beats per minute (bpm). When outlier-removed heart rate data 504 exceeds this maximum heart rate threshold, alert 508 is generated and remains in a high or alerted state until outlier-removed heart rate data 504 decreases below maximum heart rate threshold 506a. As shown in the FIG. 5A, an increased risk indicator—in the form of alert 508—appears before a heart failure event 510.

In the embodiment shown in FIG. 5B, heart rate data 502 remains the same, as does the median or outlier-removed heart rate data 504. However, rather than utilizing a simple heart rate threshold (e.g., 75 beats per minute), the median heart rate 504 is compared to a percentage of the age-predicted maximum heart rate (APMHR) (e.g., 50% of APMHR) 512. In one embodiment, APMHR is calculated as two-hundred and twenty (220) minus the patient's age, but other metrics may be used to tailor this value to the particular patient being monitored. Once again, an alert or flag 514 is set in response to the median heart rate 504 exceeding this value. Once again, an increased risk indicator—represented by alert 514—appears before heart failure event 510.

In the embodiment shown in FIG. 5C, rather than compare the media heart rate data 504 to a threshold, a plurality of heart rate bands B are defined and a risk number R is assigned to each band B. A count is kept over a given period of time of the number of heart rates that fall within each of the heart rate bands. In one embodiment, the count associated with each band is multiplied by the risk number, and the sum of this operation performed over each band is used to generate an aggregate risk number 516—shown in the lower graph of FIG. 5C. The aggregate risk number 516 is compared to a threshold value (e.g., 2), and if it exceeds the threshold value triggers alert 518. For example, in one embodiment, six bands BNk (with k=6) are defined with respect to various heart rate values and each is assigned a risk value R as follows: B={[<40 bpm], [40-60 bpm], [60-70 bpm], [70-80 bpm], [70-80 bpm], [>80 bpm]}, R={3, 2, 0, 1, 2, 3}. A value BNk gives a count of the number of heart rate values that fall in each bin. For example, a heart rate value of 30 bpm falls in band 1, with an associated risk value of 3, while a heart rate value of 65 bpm falls into band 3, which an associated risk value of 0. Heart rate values are stored to bins over a given window, and then discarded, such that the aggregate risk number is provided with respect to a certain period of time. The aggregate risk number E_{w}—illustrated by line 516—is calculated as E_{W}=ΣₖBNk*Rk, which multiplies the count of heart rates associated with each bin of bands (BNk) by the risk value associated with each band, and sums the result. As shown in the FIG. 5C, an increased risk indicator—indicated by alert 518—once again appears before a heart failure event 510.

In the embodiment shown in FIG. 5D, the calculation of the risk value is modified from what was described with respect to FIG. 5C. Once again, a plurality of heart range bands are defined, and a count is kept over a given period of time of the number of heart rates that fall within each of the heart rate bands. In the embodiment described with respect to FIG. 5C, each band is assigned a risk value and the count of heart rates measured within each band are used in conjunction with the risk values to determine an aggregate risk value. In this embodiment, only the count of hearts rates measured in the bins associated with the maximum risk value (e.g., the bins associated with heart rates <40 bpm or >80 bpm) are counted. That is, the count of maximum risk bins is represented as BN_{Max}=BN1+BN6, where BN1 and BN6 are the counts of the number of heart rate values in the bands which the highest risk numbers, which in this example are band 1 (<40 bpm) and band 6 (>80 bpm) with R=3. The heart rate risk value can be calculated by dividing the value BN_{Max} by the total number of heart rate values measured in the time window. If this risk number exceeds a threshold, e.g., 0.6, then alert or indicator flag is asserted. Once again, an increased risk indicator—illustrated by line 522—appears before a heart failure event 510. With respect to the embodiments shown in FIGS. 5C and 5D, the heart rate bands can be personalized to a patient based on age, medication, typical activity levels, medical history, and other factors. Further, a window's risk measure R_{w} can equal the proportion of {HR(i)} in the highest risk bands.

In some examples, if the variability (e.g., standard deviation or inter-percentile deviation) of the heart rate values derived from the median heart rate {HR(i)} is greater than a threshold, such as 100 ms, then patient CHF risk is low. If the variability of the heart rate values derived from the median heart rate {HR(i)} is below a threshold, such as 45 ms, then patient CHF risk increases.

In some examples, the continuous risk measure variables can be assigned to each moving window instead of a flag. Trends in the measures such as increased trends in the expected risk number or threshold crossing can then be used to monitor and determine CHF risk.

Combining comparison outputs to create composite risk factors—such as composite risk factors 308 discussed with respect to FIG. 3B—in some examples may comprise combining comparison outputs generated from one or more physiological values. Composite risk factors can be created using device-derived values, pre-monitoring information, or a combination thereof. In some examples, individual filtered physiological values may be combined to determine patient CHF risk. In other examples, filtered physiological values may be combined with unfiltered physiological values to determine patient CHF risk. For example, filtered heart rate values may be combined with unfiltered activity intensity value trends to determine patient CHF risk. In other examples, one or more unfiltered physiological values may be combined to determine patient CHF risk. For example, unfiltered activity intensity value trends may be combined with arrhythmia detection values to determine CHF risk. For each filtered or unfiltered physiological value, one or more risk values can be determined for a given time window, or flag counts can be accumulated for one or more time windows. Combining can comprise totaling flag counts for one or more physiological values over one or more time windows. Combining can comprise averaging risk values for one or more physiological values over one or more time windows.

In some examples, a CHF status or a likelihood of re-hospitalization after discharge is computed as the product of the base likelihood from in-hospital clinical measurements (e.g., labs) and medical history and the event likelihood from the bioimpedance, accelerometer, and heart rate-based metrics presented above. For example, the PiiX-based real-time measurements can be used as serial diagnostics to improve the accuracy of a likelihood of re-hospitalization based on in-hospital clinical measurements (e.g., labs) and medical history. This framework is useful when certain sensors are not available (e.g., improper bioimpedance due to poor contact) or not appropriate (e.g., heart rate values in a patient with a pacer). Similarly, sensors which are sensitive during an in-hospital phase can be identified and only measurements and likelihood of re-hospitalization information from those identified sensors will be considered in computing a likelihood of re-hospitalization. For example, if a patient is mostly sedentary, the accelerometer values will not be considered. For example, if the bioimpedance dynamic response is not visible in the hospital phase, those bioimpedence values will not be considered in computing a likelihood of re-hospitalization.

Pre-test likelihood of hospitalization factors are, in many embodiments, used to condition physiological values. In some examples, it is possible to add other sources of diagnostic information. For example, a patient may be tested for B-type natriuretic peptide (BNP) during routine hospital visits, and lab results may be incorporated into a personalized risk determination method. Additionally, pre-test likelihood of hospitalization factors can be used to interpret physiological values. For example, a patient with a high body mass index (BMI) will be expected to have higher than normal bioimpedance measurements, as compared to patients with lower BMIs.

In some examples only the PiiX sensor metrics-based CHF event-likelihoods will be provided to an attending physician, such as one having expert knowledge of the disease and the individual patient. The physician can combine this knowledge with the PiiX sensor information to triage patients as they deem fit. For example, a pre-test likelihood of re-hospitalization may be combined with sequential diagnoses from data gathered by, for example, a Piix device to generate a post-test likelihood of re-hospitalization due to, for example, CHF. Such a method also allows for the physicians to discount or fortify the knowledge provided by the metrics in specific patients. For example, a physician can choose not to consider bioimpedance in patients with COPD or renal failure and only consider heart rate metrics.

Comparison outputs from a plurality of physiological values may be combined to create composite risk factors. For example, indicator flags (comparison outputs) for each of the physiological parameters can be created as follows. Let {t1, p1}, {t2, p2}, . . . {tN, pN} denote the series of time instance and physiological values. If the physiological parameter meets a detection criterion at time tN, the indicator flag is asserted at that time, else it is not. A detection criterion may be a high or low threshold, for example. An alternative to individual flag counts can be asserting a weighted flag based on the amount a threshold is crossed. For example, a weighted indicator flag=1, may be asserted if a measured physiological value exceeds a high threshold or falls below a low threshold by an increment of, for example, 10%, a weighted indicator flag=2, may be asserted if a measured physiological value exceeds a high threshold or falls below a low threshold by an increment of, for example, 20%, and so on.

The physiological values are compared to either a patient-specific baseline or absolute threshold values to create indicator flags. For example, 5 different indicator flags may be created:
1. Bioimpedance indicator flags can be created by comparing measured values to values measured over a historical time-window. If the bioimpedance-based value, for example fluid status, for the current time window is a certain percentage above or below the mean of the historical values, a bioimpedance indicator flag is asserted. The flag can be de-asserted a number of house the percentage difference decreases. For example, compare the median of a 48-hour window of bioimpedance fluid status values with the median of the trailing 48-hour window. If the percentage difference between the values >10%, then a bioimpedance indicator flag is asserted. The indicator flag can be de-asserted 24 hours after the percentage difference is <10%.
2. Heart rate indicator flags can be created based on methods described above, wherein a risk number is created for a moving window. If the moving-window risk number exceeds a threshold (e.g., 2) then a HR indicator flag is asserted. The indicator flag can be de-asserted a number of hours, e.g., 24 hours, after the risk number drops below the given threshold.
3. Heart rate indicator flags can also be created based on other methods described above, wherein risk is measured based on heart rate variability as compared to a threshold. An indicator flag can be asserted if heart rate variability exceeds a threshold, e.g., 100 ms, and the flag can be de-asserted a number of hours, e.g., 24 hours, after the heart rate variability drops below the given threshold.
4. Combined indicator flags, for example breath rate and posture-based indicator flags, can be created in a manner similar to the heart rate indicator flags described based on a moving window breath-rate risk-number. A moving window breath-rate risk-number can be determined (e.g., define number of bands k=3, and risk bands B={[<6 brpm], [6-20 brpm], [>20 brpm]}, and risk value assigned per band R={2, 1, 2}). Breath rate indicator flags can be asserted and de-asserted as described above. A posture-based flag can be asserted if a patient spends less than a number of hours per day in a rest state. For example, an indicator flag can be asserted if a patient spends less than 40% of the day in a supine rest posture. A combined indicator flag can be asserted when either the breath rate indicator flag or the posture-based indicator flag are asserted. Such combined indicator flags may be based on a plurality of individual indicator flags.
5. Arrhythmia risk flags can be created based on, for example, atrial fibrillation (AF) burden. For example, if the 24-hour AF burden is greater than a given threshold and at least one AF episode has been identified by manual over-read in that 24 hour window, an AF burden indicator flag can be asserted.

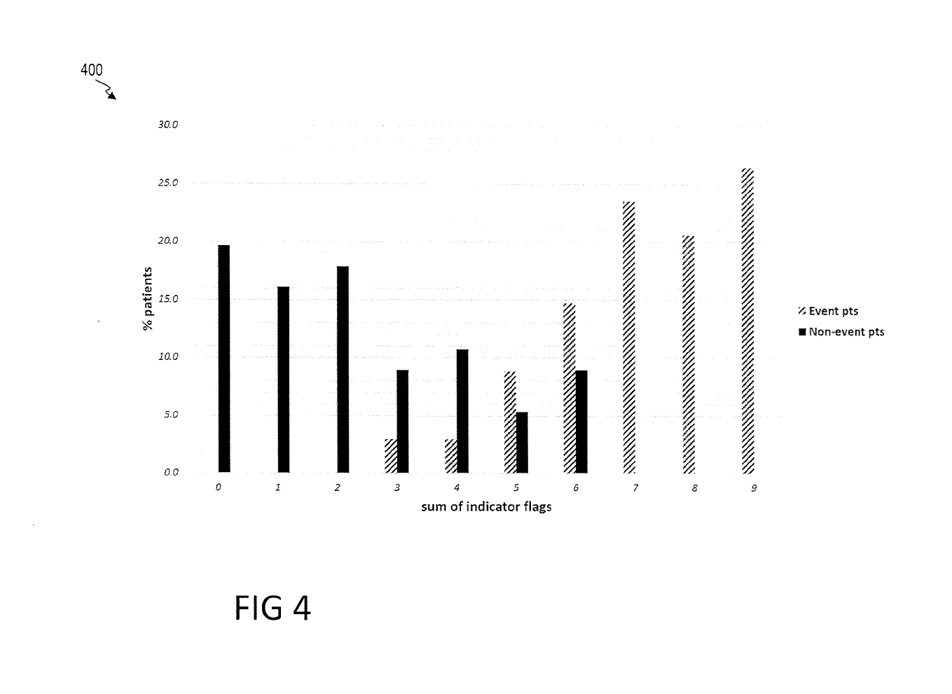

Any number of indicator flags may be created, and each created flag can correspond to any given physiological value or combination of physiological values. Once a number of indicator flags, in this example 5, have been created, let {t, F1, F2, F3, F4, F5}1, {t, F1, F2, F3, F4, F5}2, . . . {t, F1, F2, F3, F4, F5}N denote the 5 indicator flags at time t1, t2, . . . tN. A composite device-based score can be created by summing the risk flags over time. For example, a composite score={t, ΣF}1, {t, ΣF}2, {t, ΣF}N. By comparing the ΣF values to a distribution of ΣF vs. heart failure event occurrence derived from a development dataset, a physician can correlate the risk of heart failure (i.e., probability of occurrence derived from a similar population) based on the value of ΣF. FIG. 4 shows a sample distribution 400 derived with indicator flags that can take value of 0, 1 or 2 for non-heart failure event patients and heart failure event patients.

A second composite device-based risk score can be created based on binary indicator flags. A positive and negative likelihood ratio L for each physiological indicator flag can be derived from the development dataset. The positive likelihood ratio is the likelihood of a heart failure while an indicator flag is asserted, and negative likelihood ratio is the likelihood that no heart failure event will occur while an indicator flag is not asserted. The composite score=the product of the likelihood ratios for each physiological indicator.

Patient-specific information can be used to tailor composite device-based risk factors. In tailoring composite risk factors includes, risk factors used must be compatible with a patient's specific medical characteristics. For example, Table 1. displays examples of how compatible indicator flags can be selected based on patient history:

TABLE 1
Physiological Risk Factor Compatibility with Patient-Specific Characteristics

| | | | | Combined Breath | |
|---|---|---|---|---|---|
| | | | Heart Rate | Rate and | |
| Patient | Bioimpedance | Heart Rate | Variation | Accelerometer | AF burden |
| Condition | indicator flag | Indicator Flag | Indicator Flag | Indicator Flag | indicator flag |
| Systolic Heart | Yes, with | Yes | Yes | Yes | No |
| Failure | threshold = −10% | | | | |
| Diastolic Heart | Yes, with | Yes | Yes | Yes | Yes |
| Failure | threshold = −5% | | | | |
| Pacer Present | Yes | No | No | Yes | Yes |
| Pacer Absent | Yes | Yes | Yes | Yes | Yes |

As shown above, if a patient uses a pacer, CRT, or ICT, heart rate and heart rate variability indicator flags should not be considered, for example. For systolic heart failure patients, compute the Bioimpedance indicator flag with a threshold of −10%, while for diastolic heart failure patients a threshold of −5% should be used to capture fast changes in fluid status. AF burden should be used as a risk factor for heart failure in diastolic heart failure patients, but not in systolic heart failure patients. The patient-specific physiological parameters can be combined either as a sum of indicator flags or as a product of likelihood ratios to provide a likelihood of heart failure given these physiological parameters.

Physicians can also choose to combine the physiological parameters they deem appropriate for each patient to create a device-based composite risk factor. In this case, the system provides a combined likelihood of heart failure given the physician chosen physiological parameters. The composite risk score provides a likelihood of heart failure given the chosen physiological parameters. It can also be combined with the pre-monitoring patient odds of heart failure based on factors such as patient demographics, medical history, laboratory results, and physician judgment in order to compute the patient's odds of heart failure re-hospitalization.

Using the methods and devices described herein, a likelihood of 30-day CHF re-hospitalization can be monitored using several methods. These methods can also be implemented for longer or shorter time periods, such as 14 days or 60 days. One method provides for a likelihood of 30-day re-hospitalization based on clinical data and data collected by a device such as a Piix device wherein statistical models such as logistic regression can be used.

One method provides for a likelihood of CHF re-hospitalization 8-37 days post-discharge based on clinical data collected during a patient's heart failure hospitalization and data collected by a device such as a Piix device one week after discharge. In this method, statistical models such as Cox proportional hazards can be used.

One method provides for a likelihood of 30-day CHF re-hospitalization based on data collected daily by a device such as a Piix device wherein statistical models such as logistic regression, discrete-time survival analysis, or Cox proportional hazards with time-dependent variations can be used,

One method provides for a likelihood of CHF re-hospitalization for a window of N days given previous data collected by a device such as a Piix device. For example, the method may provide a likelihood of re-hospitalization 10-14 days after patient discharge based on data collected by a device such as a Piix device collected 3-10 days after patient discharge, or may provide a likelihood of re-hospitalization 12-15 days after patient discharge based on data collected by a device such as a Piix device collected 5-12 days after patient discharge. Generally, the method may provide a likelihood of re-hospitalization from day x+7*n to day x+7*(n+1) after patient discharge based on data collected by a device such as a Piix device collected from day x to day x+7*n after patient discharge, where x is a number of days and n is an integer.

Methods disclosed herein can also provide for enhanced patient screening, aggregation of data, and risk stratification efficacy verification. For example, only patients with improved CHF statuses may be selected for risk stratification to create a more reliable sub-population that can be risk stratified for re-hospitalization based on data collected by a device such as a Piix device. In another example, separate methods can be used for systolic and diastolic heart failure patients to more quickly identify changes and trends in, for example, diastolic heart failure patients.

Methods disclosed herein can provide for displaying of heart failure information. For example, the methods and physiological values described herein may be displayed, along with risk determination and stratification measures, for a physician to act upon. Composite risk factors may be displayed as-is to provide a direct measure of hospitalization risk. Alternatively, device-derived composite risk factors can be combined with pre-monitoring odds of hospitalization, based on patient demographics, medical history, laboratory work, or physician judgment, to get a post-monitoring risk of hospitalization.

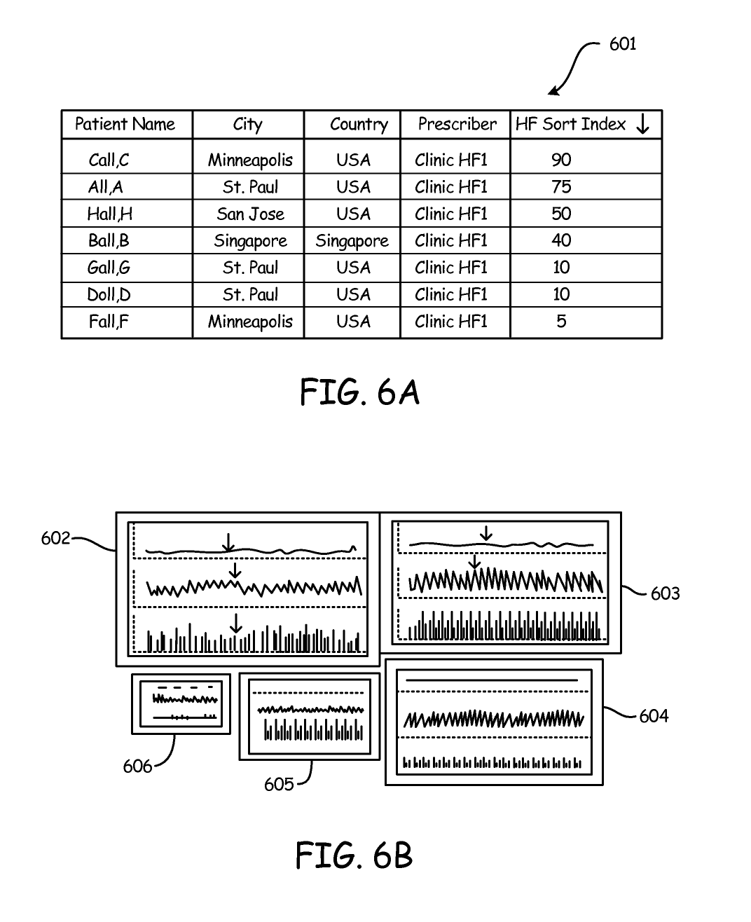

FIGS. 6A and 6B illustrate exemplary displays provided to monitor heart failure information. In the embodiment shown in FIG. 6A, sortable patient display 601 provides information that includes but is not limited to patient's name, address, prescriber, and CHF re-hospitalization risk. As indicated, patient display 601 is provided in a table format that is sortable by one or more of the displayed values. For example, in the example shown in FIG. 6A the table is sorted by CHF re-hospitalization risk, which is displayed numerically for each patient and is illustrated on the right-side column. By sorting the patients by the numeric risk status, it allows hospital personnel to quickly identify and respond to CHF conditions. In other embodiments, the display shown in FIG. 6A may include other sortable metrics provided by the Piix device, such as one or more of bioimpedance values, ECG values, and 3-axis accelerometer values, for example. Patient-wise sorting metric enables information from a device, such as a Piix device, and/or other information such as patient demographics, lab work, and prescribed medications, to be displayed in a sortable manner. For example, the information may be sorted by risk level, or by device-derived composite risk factors. Separate displays can be created per physician or per clinic to allow a physician to see a personalized list of patients sorted in order of CHF re-hospitalization risk. For example, an index to enable sorting=[(Delta Bioz(+ve/−ve adjusted)/4 hrs)*0.7+(Avg HRV/4 hrs*0.1)+(Activity Index/4 hrs*0.1)+(Avg Respiration rate/4 hrs*0.1)]. The sorted patient display can be a text list, as shown in the FIG. 6A, or a visual display such as that shown in FIG. 6B.

In the embodiment shown in FIG. 6B, collected data or calculated risk values are displayed according to dynamic tiles sizes and colors. For example, a patient identified with the highest CHF risk index may be displayed in a tile having a greater size than other tiles, and may be color-coded (e.g., red) in order to draw attention to the tile. Likewise, patients identified with lower CHF risk indexes may be displayed in tiles having relatively smaller sizes (e.g., tiles 603 and 604) and may be color-coded as such (e.g., orange), while patients identified with the lowest CHF risk indexes may be displayed in tiles having even smaller sizes (e.g., tiles 605 and 606) and may be color-coded as such (e.g., green). In this way, it makes it very easy for hospital personnel to identify those patients at the highest risk level of CHF.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

What is claimed is:

1. A method for determining a health status of a patient, the method comprising:
determining one or more physiological values of the patient;
determining a rest state of the patient based on the one or more physiological values;
filtering one or more of the physiological values based on the patient rest state; and
determining a congestive heart failure (CHF) risk of the patient based on the one or more filtered values.

2. The method of claim 1, wherein determining one or more physiological values comprises obtaining electrocardiogram (ECG) recordings, bioimpedance measurements, and 3-axis accelerometer measurements.

3. The method of claim 1, wherein one or more physiological values include fluid status, heart rate, heart rhythm, breath rate, breath volume, body posture, activity intensity, and activity duration.

4. The method of claim 1, wherein determining a patient rest state comprises:
obtaining 3-axis accelerometer measurements wherein an x-axis and a y-axis are on the patient's body plane and a z-axis is perpendicular to the body plane.

5. The method of claim 4, wherein determining a patient rest state further comprises determining one or more of:
a distribution range of z-axis measurements;
a relative magnitude of the z-axis measurements to the x-axis measurements and the y-axis measurements;
a relative magnitude of the z-axis measurement to a sum of the squared x-axis measurements and squared y-axis measurements;
a relative magnitude of the y-axis measurements to the x-axis measurements and the z-axis measurements; and
a relative magnitude of the y-axis measurement to a sum of the squared x-axis measurements and squared z-axis measurements.

6. The method of claim 1, wherein determining whether a patient is at rest further comprises determining one or more of:
a patient sleep schedule;
sleep schedule data provided by the patient; and
a time of day.

7. The method of claim 1, wherein filtering physiological values comprises one or more of:
disregarding heart rate measured while the patient is not at rest;
disregarding breath rate measured while the patient is not at rest;
disregarding breath volume measured while the patient is not at rest; and
disregarding activity intensity measured while the patient is at rest.

8. The method of claim 1, wherein determining a CHF risk comprises comparing filtered physiological values to baseline physiological values to generate a comparison output.

9. The method of claim 8, wherein a baseline physiological values are determined based on one or more of patient demographics, physiological values determined during a patient hospitalization, historic physiological values determined after a patient hospitalization, clinical lab results, and patient medical history.

11. The method of claim 8, wherein a comparison output comprises one or more of an occurrences flag indicating that filtered physiological value is below or exceeds a baseline physiological value, and a comparative percentage to which a filtered physiological values is below or exceeds a baseline metric.

12. The method of claim 11, wherein determining CHF risk comprises determining an occurrence frequency at which an occurrence flag is generated for a given time window.

13. The method of claim 12, wherein determining CHF risk further comprises determining a trend in occurrence frequencies for successive time windows.

14. The method of claim 11, wherein determining CHF risk further comprises determining a trend in the magnitude of comparative percentages for a given or successive time windows.

15. The method of claim 8, further comprising creating a composite risk factor by combining a plurality of comparison outputs.

16. A device for monitoring and determining a risk of congestive heart failure (CFH) hospitalization, the device comprising:
a 3-axis accelerometer configured to measure patient physiological values;
one or more bioimpedance sensors configured to measure patient physiological values;
an electrocardiogram configured to measure patient physiological values;
one or more processors to receive the measured physiological values.

17. The device of claim 16, further comprising a patch capable of removably attaching to the body of a patient.
